(19) United States
(12) Patent Application Publication
(10) Pub. No.: US 2022/0326217 A1
(43) Pub. Date: Oct. 13, 2022

(54) COMPOSITIONS AND METHODS FOR DETECTING ALLERGEN REACTIVE TH2 CELLS

(71) Applicant: Benaroya Research Institute at Virginia Mason, Seattle, WA (US)

(72) Inventors: Eric E. Wambre, Seattle, WA (US); Justine J. Calise, Seattle, WA (US)

(73) Assignee: Benaroya Research Institute at Virginia Mason, Seattle, WA (US)

(21) Appl. No.: 17/640,548

(22) PCT Filed: Sep. 4, 2020
(86) PCT No.: PCT/US2020/049501

§ 371 Date: Mar. 4, 2022

Related U.S. Application Data
(60) Provisional application No. 62/897,091, filed on Sep. 6, 2019.

(51) Int. Cl. G01N 33/50; G01N 33/68; A61P 37/08

(52) U.S. Cl. CPC G01N 33/505; G01N 33/6893; A61P 37/08; G01N 2333/7155; G01N 2800/24; G01N 2800/52

(57) ABSTRACT

Provided are methods and compositions for labeling an allergen-specific pathogenic CD4+ T-cell. The method can comprise contacting a cell population comprising CD4+ T cells with a suspected allergen to provide a challenged cell population, contacting the challenged cell population, or a subpopulation thereof, with a first molecule that specifically binds to a biomarker for an allergen-specific pathogenic T cell, wherein binding of the first molecule to the biomarker on a CD4+ cell indicates the cell is an allergen-specific pathogenic CD4+ T cell, and detecting binding of the first molecule to a CD4+ cell, wherein binding to the cell indicates the cell is an allergen-specific pathogenic CD4+ T cell. The method is applicable to monitoring the presence of allergen-specific pathogenic CD4+ T cells and/or efficacy of immunotherapy for allergies in a subject.

### CROSS-REFERENCES TO RELATED APPLICATIONS

This application claims the benefit of U.S. Patent Application No. 62/897,091, filed Sep. 6, 2019, which is incorporated herein by reference in its entirety.

### STATEMENT OF GOVERNMENT LICENSE RIGHTS

This invention was made with Government support under 5U19AI35817 and AI108839 awarded by the National Institutes of Health. The Government has certain rights in the invention.

### BACKGROUND

As part of their specialization, CD4+ effector T cells acquire functional and phenotypic characteristics to specifically respond against pathogens. Within different T helper (T_{H}) cell subsets, the T helper type 2 (T_{H}2) cell subset is characterized by the production of IL-4, IL-5, IL-9 and IL-13 cytokines, which promote both IgE- and eosinophil-mediated immune responses. Although T_{H}2 cells were initially considered to be a homogeneous subset, their functional heterogeneity is now appreciated, including T_{H}2-driven pathology which can be determined by T_{H}2 subpopulations. Allergen-specific T_{H}2 cells play a central role in initiating and orchestrating the allergic and asthmatic inflammatory response pathways.

A source of heterogeneity among CD4+ T cell subsets is T cell-surface biomarker expression, which determines their differentiation states, effector functions, and migratory capacity. Utilization of cellular biomarkers can provide useful information regarding disease status, patient stratification, or response to therapy. T_{H}2A cells represent a subset of pathogenic terminally differentiated T_{H}2 cells present only in allergic individuals. T_{H}2A cells can be identified based on differential expression levels of cell surface biomarkers such as CD4, CD27, CRTH2, and CD161. Presence of T_{H}2A cells does not indicate the specific type of allergy afflicting the individual.

While allergen-specific T_{H}2 cells play a central role in the allergic and asthmatic inflammatory response pathways, there are limitations. One major factor limiting the use of such atopic disease-causing T cells as both therapeutic targets and clinically useful biomarkers is the lack of an accepted methodology to determine specificity. Thus, despite advances in the art, a need remains for reliable strategies to generate, detect, and isolate T cell subsets indicative of a subject's sensitivity to an allergen of interest. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of specifically labeling an allergen-specific pathogenic T cell (or a population thereof). The method comprises contacting a cell population comprising CD4+ T cells with a suspected allergen to provide a challenged cell population; contacting the challenged cell population, or a subpopulation thereof, with a first molecule that specifically binds to a biomarker for an allergen-specific pathogenic T cell, wherein binding of the first molecule to the biomarker on a CD4+ cell indicates the cell is an allergen-specific pathogenic CD4+ T cell; and detecting binding of the first molecule to a CD4+ cell, wherein binding to the cell indicates the cell is an allergen-specific pathogenic CD4+ T cell.

In another aspect, the disclosure provides a method of determining whether a subject is allergic to a suspected allergen. The method comprises contacting tissue, whole blood, or peripheral blood mononuclear cells (PBMCs) obtained from a subject with a suspected allergen to provide challenged PBMCs; contacting the challenged PBMCs, or a subpopulation thereof, with a first molecule that specifically binds to a biomarker for an allergen-specific pathogenic CD4+ T cell, wherein binding of the first molecule to the biomarker on a CD4+ cell indicates the cell is an allergen-specific pathogenic CD4+ T cell, detecting binding of the first molecule to a CD4+ cell, wherein binding to the cell indicates the cell is an allergen-specific pathogenic CD4+ T cell; and determining the presence of an allergen-specific pathogenic CD4+ T cell, wherein the presence of an allergen-specific pathogenic CD4+ T cell indicates the subject is allergic to the allergen, and the absence of an allergen-specific pathogenic CD4+ T cell indicates the subject is either not allergic or has a degree of sensitization to the allergen.

In some embodiments, the method further comprises treating the subject's allergic condition. In some embodiments, treating the subject comprises administering immunotherapy.

In another aspect, the disclosure provides a method of monitoring the presence of allergen-specific pathogenic CD4+ T cells in a subject allergic to the allergen. The method comprises contacting tissue, whole blood, or peripheral blood mononuclear cells (PBMCs) obtained from the subject with the suspected allergen to provide challenged PBMCs; contacting the challenged PBMCs, or a subpopulation thereof, with a first molecule that specifically binds to a biomarker for an allergen-specific pathogenic CD4+ T cell, wherein binding of the first molecule to the biomarker on a CD4+ cell indicates the cell is an allergen-specific pathogenic CD4+ T cell; detecting binding of the first molecule to a CD4+ cell, wherein binding to the cell indicates the cell is an allergen-specific pathogenic CD4+ T cell; and determining the relative abundance over time of allergen-specific pathogenic CD4+ T cells, wherein a decreased abundance of allergen-specific pathogenic CD4+ T cells indicates the subject has a degree of desensitization to the allergen.

In some embodiments, at least one of the two or more time points during immunotherapy, or other therapy, occurs during or after treatment for the subject's allergic condition. In some embodiments, the eventual absence of allergen-specific pathogenic CD4+ T cells indicates the subject is no longer allergic to the allergen or desensitized to the allergen.

In another aspect, the disclosure provides a method of monitoring the efficacy of the immunotherapy of a subject allergic to an allergen. The method comprises performing the following at one or more time points during immunotherapy: contacting tissue, whole blood, or peripheral blood mononuclear cells (PBMCs) obtained from a subject with a suspected allergen to provide challenged PBMCs; contacting the challenged PBMCs, or a subpopulation thereof, with a first molecule that specifically binds to a biomarker for an allergen-specific pathogenic CD4+ T cell, wherein binding of the first molecule to the biomarker on a CD4+ cell indicates the cell is an allergen-specific pathogenic CD4+ T cell; detecting binding of the first molecule to a CD4+ cell, wherein binding to the cell indicates the cell is an allergen-specific pathogenic CD4+ T cell; and determining the relative abundance over time of allergen-specific pathogenic CD4+ T cells, wherein a decreased abundance of allergen-specific pathogenic CD4+ T cells indicates efficacy of the immunotherapy.

In another aspect, the disclosure provides a kit that comprises a molecule which specifically binds to a biomarker for an allergen-specific pathogenic CD4+ T cell.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a series of illustrative scatter plots from flow cytometry analyses of allergen-specific CD4+ T cells derived from peripheral blood mononuclear cells (PBMCs) obtained from allergenic individuals. FIG. 1 illustrates ST2 and IL-17RB co-expression on allergen-specific pathogenic cells.

FIG. 2 is a series of illustrative scatter plots from flow cytometry analyses of allergen-specific CD4+ T cells derived from peripheral blood mononuclear cells (PBMCs) obtained from individuals allergic to peanuts as well as non-allergenic individuals. FIG. 2 illustrates ST2 expressing allergen-specific pathogenic CD4+ T cells are confined to atopic individuals.

FIG. 3 is a series of illustrative flow cytometry scatter plots and histogram analyses of allergen-specific CD4+ T cells derived from peripheral blood mononuclear cells (PBMCs). T cell activation resulted from pooled allergen stimulation comprising peanut, pollen, and dust mite allergens. FIG. 3 illustrates that ST2 expression on human T_{H}2A cells requires prior T cell receptor triggering.

FIG. 4 is a series of illustrative dot plots from flow cytometry analyses of peripheral blood mononuclear cells (PBMCs) obtained from an individual allergic to alder pollen. Subpopulations of PBMC's that were CD4+CD45RA− were obtained and challenged with alder pollen extract to detect cells allergen-reactive pathogenic cells through the ST2 expression assay in comparison with PBMCs not challenged with the allergen extract.

FIG. 5 is a series of illustrative dot plots from flow cytometry scatter plot analyses of total CD4+ T cells derived from peripheral blood mononuclear cells (PBMCs). FIG. 5 illustrates untreated total CD4+ T cell populations, in comparison with total CD4+ T cell populations treated with pollen extract. The cells were obtained from the same individual allergic to alder pollen as the cells assessed for FIG. 4.

FIG. 6 is a series of illustrative dot plots from flow cytometry scatter plot analyses of T cells derived from peripheral blood mononuclear cells (PBMCs). FIG. 6 illustrates untreated cell populations, in comparison with cells treated with a combination dust mite and pollen extract, in an individual allergic to dust mite and pollen.

FIG. 7 is a series of illustrative dot plots from flow cytometry scatter plot analyses of T cells derived from peripheral blood mononuclear cells (PBMCs). FIG. 7 illustrates untreated cell populations in comparison with cell populations treated with milk extract, dust mite extract, and combination peanut and walnut extracts, in an individual allergic to milk, dust mites, and nuts.

FIG. 8 is a series of illustrative dot plots from flow cytometry scatter plot analyses of T cells derived from peripheral blood mononuclear cells (PBMCs). FIG. 8 illustrates cell populations treated with peanut extract in comparison with cell populations treated with a combination of alder and grass extracts in an individual allergic to alder and grass pollen but not allergic to peanuts.

FIG. 9 is a series of illustrative dot plots from flow cytometry scatter plot analyses of T cells derived from peripheral blood mononuclear cells (PBMCs). FIG. 9 illustrates cell populations not treated with allergen or extract, cell populations treated with pool inhalant allergen extracts, and cell populations treated with peanut extract, in an individual not allergic to pool inhalants or peanuts.

### DETAILED DESCRIPTION

This disclosure generally provides methods, related systems, and reagents for specifically labeling, detecting, and quantifying subpopulations of allergen-specific pathogenic CD4+ T cells. These methods are useful for further applications. For example, the methods are useful in determining the allergic condition of a subject to any allergen of interest, monitoring allergen-specific pathogenic CD4+ T cells, and monitoring the efficacy of immunotherapy to address an allergic condition. This disclosure is based on the characterization of a subset of human memory T_{H}2 cells confined to atopic individuals and includes all allergen-specific T_{H}2 cells. As described in more detail in Example 1 below, an ex vivo method using peptide-MHC Class II tetramers was employed to detect and label allergen-specific pathogenic CD4+ T cells after ex vivo exposure of collected peripheral blood mononuclear cells (PBMCs) to an allergen of interest. It was determined that subsets of the allergen-specific pathogenic CD4+ T cells were differentiated. This subset of cells was characterized by the expression of ST2 and IL-17RB, and exhibited numerous functional attributes distinct from conventional T_{H}2 cells. ST2 is an IL-33 receptor upregulated upon PBMC exposure to allergens. Further analysis reveals involvement of these cells in a distinct pathway associated with the initiation of pathogenic responses to allergen; elimination of these cells is indicative of an immunotherapy clinical response. ST2 detection of allergen-specific pathogenic CD4+ T cells has been found to serve as a biomarker for allergen specificity in the T cells. Screening for expression of ST2, as an alternative to existing approaches for detecting allergen-specific pathogenic CD4+ T cells, has provided proof of concept that such a screen could reliably identify allergen-specific pathogenic CD4+ T cells for further differentiation analysis. It is shown the method could be applicable to a variety of unrelated allergens of interest, demonstrating the functionality of the platform method across a multitude of allergic conditions.

In accordance with the foregoing, in one aspect, the present disclosure provides a method of specifically labeling an allergen-specific pathogenic CD4+ T cell. The method comprises contacting a cell population comprising T cells with an allergen of interest to provide a challenged cell population, then contacting the challenged cell population with a molecule that specifically binds to a biomarker for an allergen-specific pathogenic CD4+ T cell, wherein binding of the molecule to the biomarker on a cell indicates that cell is an allergen-specific pathogenic CD4+ T cell.

Various facets of the innovation are described below.

Cell population

The cell population can be any heterogeneous or homogenous population of cells that comprises or consists of T lymphocytes (“T cells”). For instance, the cell population can comprise PBMCs, which can be routinely isolated from blood samples obtained from a subject. In other embodiments, the cell population can be in a whole blood sample without any substantial isolation or further processing. In some embodiments, the cell population can be in various subpopulations of blood cells including, for example, white cell populations, T cell populations, or T helper cells, which can typically be identified as expressing CD4 (CD4+) on their surface.

The cell population can be freshly acquired, previously frozen, or stored, to facilitate convenience.

In certain embodiments, the method further comprises obtaining a biological sample (e.g., whole blood) from the subject.

Challenge with suspected allergen

The present disclosure addresses various methods for labelling, detecting, quantifying, and monitoring active T cells specific for an allergen or suspected allergen. As demonstrated in the Examples, the methods described herein are not limited by any particular allergen but can be widely applied as a platform to address any particular allergen of interest. Thus, the allergen can be a food allergen or environmental allergen. An exemplary, non-limiting list of food allergens encompassed by the present methods include, but are not limited to, peanut, soy, wheat, dairy, eggs, tree nuts (e.g., almonds, cashews, walnuts, and the like), fish (e.g., bass, cod, flounder, and the like), and shellfish (e.g., crab, lobster, shrimp, and the like). An exemplary, non-limiting list of environmental allergens include pollens (e.g., grass, tree, and the like), dust mites, pet and other animal dander, mold and mildew, smoke and ash (e.g., from cigarettes), and chemical (e.g., pool inhalants).

The cell population can be challenged with a suspected allergen in a variety of ways. The suspected allergen can be in a crude extract or isolate, such as from ground and liquefied peanut. Alternatively, specific proteins or portions thereof can be used, such as those substantially or completely isolated or purified. Exposure to the allergen can be brief or extensive, to provide sufficient time for allergen specific cells to bind to their cognate allergen epitope and be differentiated thereby. Exposure duration can be as short as 15 minutes. As described in the Examples, challenges were successfully performed within a one-hour duration, but can be extended longer if necessary. Once exposed to the allergen, the cell population is generally referred to as a challenged cell population. In some embodiments, the challenge occurs ex vivo. It will be apparent that in some embodiments, the challenged cell population contains cells with different developmental stages and/or expression patterns that differentiate them from the corresponding cells in the pre-challenged cell population, whether circulating in the subject's body or in a biological sample obtained from the subject.

### Detecting allergen-specific pathogenic CD4+ T cells

Typically, T cells specific for a particular allergen can be identified or isolated using molecules which present specific allergen epitopes recognized by the T cells. For example, ex vivo methods employing peptide-MHC class II tetramers have been used to label and enrich for T cells specific for a particular allergen. See Wambre, E., et al., “Differentiation stage determines pathologic and protective allergen-specific CD4+ T-cell outcomes during specific immunotherapy,” J. Allergy Clin. Immunol. 129(2):544-551 (2012) and Wambre, E., et al., “Specific immunotherapy modifies allergen-specific CD4+ T-cell responses in an epitope-dependent manner,” J. Allergy Clin. Immunol. 133(3):872-879 (2014), each incorporated herein by reference in its entirety. However, detecting cell specificity to the allergen using putative allergen epitopes can present several drawbacks. For example, use of the peptide-MHC class II tetramer constructed to detect and isolate allergen-specific pathogenic CD4+ T cells relies on use of the appropriate epitope, which is difficult to ensure. Furthermore, there can be multiple epitopes in any particular allergen molecule that stimulates a population of T cells and, thus, use of one or a few particular peptides conjugated with MHC class II tetramers might not capture or label all relevant allergen-specific pathogenic CD4+ T cells. Accordingly, epitope-based labelling of allergen-specific pathogenic CD4+ T cells risks providing an incomplete subpopulation of allergen-specific pathogenic CD4+ T cells.

In contrast, use of a biomarker which does not incorporate a specific epitope molecule of the allergen to assess allergen specificity would avoid such limitations. Use of such a biomarker can provide a more comprehensive screen of cells for allergen-specificity because a binary signal results after ex vivo allergen challenge. Furthermore, it has been demonstrated that assays which incorporate epitope-independent screening accurately measure allergen-specific pathogenic CD4+ T cells and are at least equivalent to detecting allergen-specific pathogenic CD4+ T cells as assays based on MHC class II tetramers conjugated with the target peptide. See Renand, A., et al., “Chronic cat-allergen exposure induces a T_{H}2 cell-dependent igG4 response related to low-sensitization,” J. Allergy Clin. Immunol. 136(6):1627-1635 (2015), incorporated herein by reference in its entirety.

Accordingly, the method disclosed herein comprises contacting a challenged cell population with a molecule that specifically binds to a biomarker on an allergen-specific pathogenic CD4+ T cell, wherein binding of the molecule to the biomarker on the T cell indicates that the cell is an allergen-specific pathogenic CD4+ T cell. Exemplary structures for the molecule are described in more detail below. The term “specifically binds” refers to the ability of the molecule to bind to the target biomarker, without significant binding to other molecules, under standard conditions known in the art. The molecule that specifically binds to the allergen-specific pathogenic CD4+ T cell biomarker can bind to other peptides, polypeptides, or proteins, but with lower affinity relative to the T cell biomarker, as determined by, e.g., immunoassays, BIAcore, or other assays known in the art. However, the molecule preferably does not cross-react with other antigens.

In certain embodiments, the method further comprises the step of determining the presence of allergen-specific pathogenic CD4+ T cells. This step comprises detecting binding of the molecule to the allergen-specific pathogenic CD4+ T cell biomarker of one or more cells within the challenged cell population, or at least within a subpopulation thereof.

In certain embodiments, the method further comprises enriching for allergen-specific pathogenic CD4+ T cells. This can be accomplished in various ways and typically leverages specific binding of the molecule to the allergen-specific pathogenic CD4+ T cell biomarker. For example, the molecule that specifically binds to the allergen-specific pathogenic CD4+ T cell biomarker can comprise a capture domain or the like that can be bound by an immobilized or immobilizable surface, such as a bead. Such exemplary bead can be immobilized in a column or have a magnetic component to facilitate isolation of the molecules and any allergen-specific pathogenic CD4+ T cells bound thereto.

Exemplary biomarkers which facilitate the detection of allergen-specific pathogenic CD4+ T cells in the challenged cell population include CD27−, CD45RB−, CCR7−, CRT_{H}2+, CCR+8, CD7−, CD49b+, CD49d+, CD161+, ST2+, IL-17RB+, HPGDS+, and CD200R+. For any such biomarker, cell surface biomarker presentation is indicative of the cells' specificity to the allergen after ex vivo challenge of the cell by the allergen. In certain embodiments, the biomarker is ST2. ST2 (also known as IL1RL1, DER4, T1 and FIT-1) is a member of the Toll-like/IL-1-receptor superfamily and has been identified as a receptor of IL-33. See Kakkar R, Lee RT. The IL-33/ST2 pathway: therapeutic target and novel biomarker. Nat Rev Drug Discov. 2008 October;7(10):827-40, incorporated herein by reference in its entirety. An exemplary, non-limiting amino acid sequence for human ST2 is provided in Genbank entry NP_057316.3, incorporated herein by reference in its entirety. Methods specifically using ST2 for a screen of pathogenic CD4+ T cells are described in more detail in the Examples. In other embodiments, the biomarker is IL-17RB. An exemplary, non-limiting amino acid sequence for human ST2 is provided in Genbank entry NP_061195.2, incorporated herein by reference in its entirety.

### Biomarker-binding Molecules

The disclosed methods incorporate use of molecules which specifically bind to the allergen-specific pathogenic CD4+ T cell biomarker in the challenged cell population. Exemplary biomarkers are discussed above. The molecules are able to specifically bind to the target biomarker under standard conditions (see Examples below) and preferably do not bind to off-target antigens or epitopes to avoid providing false positive signals.

Thus, the molecule that specifically binds to the allergen-specific pathogenic CD4+ T cell biomarker can be described as an affinity reagent. In certain embodiments, the molecule is an antibody, antibody-like molecule, receptor, aptamer, or functional antigen-binding fragment or derivative thereof. As used herein, the term “antibody” can be an antibody derived from any antibody-producing mammal (e.g., mouse, rat, rabbit, and primate including human), which specifically binds to the biomarker of interest (addressed above). Exemplary antibodies include polyclonal, monoclonal, and recombinant antibodies; multi-specific antibodies (e.g., bispecific antibodies); humanized antibodies; murine antibodies; chimeric, mouse-human, mouse-primate, primate-human monoclonal antibodies. The antigen-binding fragment molecule can be any intact antibody molecule or fragment, or derivative thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments useful in the present disclosure include Fab, Fab′, F(ab)₂, F(ab′)₂ and Fv fragments, single-chain variable antibody fragments (scFv), single-chain Fab fragment (scFab), V_{H}H fragment, V_{NAR}, nanobodies, diabodies, linear antibodies, single-chain antibody molecules, multi-specific antibodies formed from antibody fragments, and the like. A single-chain variable antibody fragment comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Antibody fragments can be produced recombinantly, or through enzymatic digestion.

Antibodies can be further modified to address various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin.

Production of antibodies can be accomplished using any technique commonly known in the art. For example, the production of a polyclonal antibody can be accomplished by administering an immunogen containing the antigen of interest (i.e., the biomarker of interest) to an antibody-producing animal. The antigen of interest (i.e., the biomarker of interest) can be administered to a mammal (e.g., a rat, a mouse, a rabbit, a chicken, cattle, a monkey, a pig, a horse, a sheep, a goat, a dog, a cat, a guinea pig, a hamster) or a bird (e.g., a chicken) so as to induce production of a serum containing a biomarker-specific polyclonal antibody. The biomarker of interest can be administered in combination with other components known to facilitate induction of a B-cell response, such as any appropriate adjuvant known in the art. Furthermore, the polyclonal antibody reagent can be further processed to remove or subtract any antibody members that have unacceptable affinity for antigens that are not the biomarker of interest. The resulting polyclonal antibody reagent will exhibit enhanced specificity for the biomarker of interest and are useful for detection and quantification purposes. Many approaches for adsorption of polyclonal antibody reagents to reduce cross-reactivity exist, are familiar to persons of ordinary skill in the art, and are encompassed by the present disclosure.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art.

Antibody fragments and derivatives that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art. Finally, the antibodies, or antibody fragments or derivatives can be produced recombinantly according to known techniques.

In other embodiments, the molecule that specifically binds to the biomarker on allergen-specific pathogenic CD4+ T cells is an aptamer. The term “aptamer” refers to oligonucleic or peptide molecules that can bind to specific antigens of interest (i.e., biomarker of interest). Nucleic acid aptamers are usually short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamer is a thioaptamer, in which some or all of the non-bridging oxygen atoms of phophodiester bonds have been replaced with sulfur atoms. This atom replacement increases binding energies with proteins and slows degradation by nuclease enzymes. In some embodiments, nucleic acid aptamers contain modified bases that possess altered side-chains which can facilitate aptamer binding to the allergen-specific pathogenic CD4+ T cell biomarker.

Peptide aptamers are protein molecules that can contain a peptide loop attached at both ends to a protamersein scaffold. The loop typically consists of between 10 to 20 amino acids, and the protamersein scaffold is typically any protein that is soluble and compact. One example of the protein scaffold is Thioredoxin-A, wherein the loop structure can be inserted into the reducing active site. Peptide aptamers can be generated or selected from various types of libraries, such as phage display, mRNA display, ribosome display, bacterial display and yeast display libraries.

In some embodiments, the molecule that specifically binds to the allergen-specific CD4+ T cell biomarker of interest is a receptor molecule or comprises a binding domain of a receptor molecule. The receptor molecule can be any receptor known that can specifically bind the biomarker of interest as the ligand.

### Detection and isolation

Labeling of cells in the challenged allergen-specific pathogenic CD4+ T cell population permits detection of the cells and profiling of the biomarkers expressed (or not expressed) on the cells. Detection can be performed by any known method. Typically, the molecule that specifically binds to the biomarker on an allergen-specific CD4+ T cell comprises a detectable moiety that emits a signal under controllable circumstances. The moiety can provide, for example, a fluorescent signal upon stimulation that permits detection and quantification of the signal (and thus binding status with the cell). In some embodiments, the detection of binding of the molecule to the cell biomarker comprises use of Fluorescence-Activated Cell Sorting (FACS) or mass cytometry (CyTOF). FACS allows cell sorting based on fluorescent signals emitted from fluorochromes attached to the cells via the molecule. CyTOF utilizes heavy metal ion tags attached to the molecule. An advantage of heavy metal ion tags is their narrower signal signatures in mass spectrometry, which avoids signal overlap between different tagged moieties.

Optionally, the disclosed method can further comprise steps of enriching for or isolating cells that possess a particular biomarker profile, as determined by the specific binding of the molecules to the allergen-specific pathogenic CD4+ T cell biomarkers. As described above, the tagging moieties (e.g., fluorochromes or heavy metal ions) can comprise a domain that binds to an immobilized binding partner. The immobilized binding partner can be immobilized on a solid substrate, such as beads in a column. Alternatively, a solid bead substrate can have magnetic properties which permit immobilization of the beads and cells bound thereto via the molecule and its binding partner. This allows the majority of cells not expressing the target biomarker to be removed from cells expressing the target biomarker.

Alternatively, cells that are tagged (e.g., stained) with the molecule can be selectively sorted by flow cytometry based on the presence or absence of the molecule specifically bound to the allergen-specific pathogenic CD4+ T cell biomarker. An advantage of flow cytometry is that cells positive for a biomarker can be detected, quantified, and isolated.

### Further applications

Elements of the above method can be adapted and applied for further analyses of allergen-specific pathogenic CD4+ T cells and potential allergic conditions.

Thus, in another aspect, the disclosure provides a method of determining whether a subject is allergic to a suspected allergen. The method comprises: contacting tissue, whole blood, or peripheral blood mononuclear cells (PBMCs) obtained from a subject with a suspected allergen to provide challenged PBMCs; contacting the challenged PBMCs, or a subpopulation thereof, with a first molecule that specifically binds to a biomarker for an allergen-specific pathogenic CD4+ T cell; detecting binding of the first molecule to a CD4+ cell, wherein binding to the cell indicates the cell is an allergen-specific pathogenic CD4+ T cell; and determining the presence of a subpopulation of allergen-specific pathogenic CD4+ T cells. The presence of an allergen-specific pathogenic CD4+ T cell subpopulation indicates the subject is allergic to the allergen, and the absence of an allergen-specific pathogenic CD4+ T cell subpopulation indicates the subject is not allergic to the allergen.

In some embodiments, the presence of allergen-specific pathogenic CD4+ T cells provides indication of an allergic state.

The method can further comprise treating the allergic subject to generate allergen tolerance. Such treatments can include immunotherapy, such as allergen-specific immunotherapy (ASIT; also referred to as allergen vaccine therapy). The theory of ASIT is that gradually increasing allergen exposure will decrease the population of reactive pathogenic T cells and increase the population of T cells which promote tolerance. Alternatively, the method can comprise advising the allergic subject to take precautionary measures with respect to the allergen.

In another aspect, the disclosure provides a method of monitoring the presence of allergen-specific pathogenic CD4+ T cells in a subject allergic to an allergen. The method comprises performing the following steps with peripheral blood mononuclear cells (PBMCs) obtained from the subject at two or more time points: contacting tissue, whole blood, or peripheral blood mononuclear cells (PBMCs) obtained from the subject with the suspected allergen to provide challenged PBMCs; contacting the challenged PBMCs, or a subpopulation thereof, with a first molecule that specifically binds to a biomarker for an allergen-specific pathogenic CD4+ T cell; and detecting binding of the first molecule to a CD4+ cell, wherein binding to the cell indicates the cell is an allergen-specific pathogenic CD4+ T cell. The relative abundance over time of a subpopulation of allergen-specific pathogenic CD4+ T cells is determined. A decreased abundance of allergen-specific pathogenic CD4+ T cells indicates the subject has become less allergic to the allergen.

Typically, at least one of the two or more time points occurs during treatment or after completion of treatment for the subject's allergic condition. Treatment can be ASIT, as described above, for the relevant allergen. In some embodiments, the steps of the method are performed a plurality of times during the course of treatment, such as 2, 3, 4, 5, 6, 7, 8, 9, 10, or more times. In some embodiments, the eventual absence (or substantial absence) of allergen-specific pathogenic T cells, as described herein, indicates the relative tolerance for the allergen. In some embodiments, this is indicative of a lack of allergy. The method can be performed at additional time-points thereafter to confirm the maintenance of such tolerance.

In yet another aspect, the disclosure provides a method of monitoring immunotherapy efficacy of a subject allergic to an allergen. The method comprises performing the following steps with peripheral blood mononuclear cells (PBMCs) obtained from the subject at one or more time points during the subject's immunotherapy: contacting tissue, whole blood, or peripheral blood mononuclear cells (PBMCs) obtained from a subject with a suspected allergen to provide challenged PBMCs; contacting the challenged PBMCs, or a subpopulation thereof, with a first molecule that specifically binds to a biomarker for an allergen-specific pathogenic CD4+ T cell; and detecting binding of the first molecule to a CD4+ cell, wherein binding to the cell indicates the cell is an allergen-specific pathogenic CD4+ T cell. The relative abundance over time of a subpopulation of allergen-specific pathogenic CD4+ T cells is determined, wherein a decreased abundance of allergen-specific pathogenic CD4+ T cells indicates immunotherapy efficacy.

The biomarkers used in identifying allergen-specific pathogenic CD4+ T cells encompassed by any of these methods are described above and not repeated here.

Embodiments of any of the methods can further comprise enriching for or isolating the allergen-specific pathogenic CD4+ T cells, as described above. As described above, such enriching or isolating steps can comprise, for example, the use of flow cytometry or magnetic beads.

In some embodiments, any of the methods can further comprise obtaining tissue or whole blood from the subject. In some embodiments, the PBMCs can be derived from the whole blood or tissue obtained from the subject. In other embodiments, the PBMCs are within a whole blood sample without isolation therefrom.

In some embodiments of the methods described herein, the molecule that specifically binds to the biomarker on an allergen-specific pathogenic CD4+ T cell is an antibody, a biomarker-binding fragment or derivative thereof, an aptamer, or receptor, as described above in more detail.

### Kit

In another aspect, the disclosure provides a kit comprising a molecule which specifically binds to a biomarker on an allergen-specific pathogenic CD4+ T cell. The molecule can be an antibody, antibody-like molecule, ligand, aptamer, or a functional antigen-binding fragment or domain thereof. As described above in more detail, the antibody-like molecule can be a single-chain antibody, a bispecific antibody, a Fab fragment, or a F(ab)₂ fragment. The single-chain antibody can be a single chain variable fragment (scFv), single-chain Fab fragment (scFab), V_{H}H fragment, V_{NAR}, or nanobody. The molecule can be configured to contain detectable moieties to provide detectable signals facilitating quantification and isolation using, for example, FACS, qPCR, or CyTOF.

The molecule can be configured to bind to any biomarker, such as the ones described above. In a specific embodiment, the molecule can be configured to bind to ST2 or IL-17RB.

The kit can also contain various culture buffers, selected allergens or allergen extracts for challenge steps, reagents (e.g., magnetic beads) for enrichment, reagents for flow cytometry, and any other reagent that can assist the performance of any of the methods described herein, and/or written indicia for performing the methods. The kit can contain or provide online access to written indicia of instructions for use of the kit in the performance of any method described herein.

### Other

Unless specifically defined herein, all terms used herein have the same meaning as they would to one skilled in the art of the present invention. Practitioners are particularly directed to Sambrook J., et al. (eds.) Molecular Cloning: A Laboratory Manual, 3rd ed., Cold Spring Harbor Press, Plainsview, N.Y. (2001); Ausubel, F.M., et al. (eds.), Current Protocols in Molecular Biology, John Wiley & Sons, New York (2010); and Coligan, J.E., et al. (eds.), Current Protocols in Immunology, John Wiley & Sons, New York (2010) for definitions and terms of art.

The use of the term “or” in the claims is used to mean “and/or” unless explicitly indicated to refer to alternatives only, or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense. “Comprise,” “comprising,” and the like indicate in the sense of “including, but not limited to.” Words using the singular or plural number also include the plural and singular number, respectively. Additionally, the words “herein,” “above,” and “below,” and words of similar import, when used in this application, refer to this application as a whole and not to any particular portions of the application. Unless stated otherwise, the term “about” implies minor variation around the stated value of no more than 10% (above or below).

Disclosed are materials, compositions, and devices that can be used for, can be used in conjunction with, can be used in preparation for, or are products of, the disclosed methods and compositions. It is understood that when combinations, subsets, interactions, groups, etc., of these materials are disclosed, each of various individual and collective combinations is specifically contemplated, even though specific reference to each and every single combination and permutation of the invention aspects cannot be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following examples are provided for the purpose of illustrating, not limiting, the disclosure.

### EXAMPLES

### Example 1

### Introduction

Results from a study of a human allergen-specific pathogenic CD4+ T cell subpopulation providing signature of an allergic disease or state are disclosed. Previous approaches included screening for allergen-specific pathogenic CD4+ T cells by tracking selective CD154 upregulation in combination with additional cell surface biomarkers including CD4, CD27, CRTH2, and CD161, or screening for allergen-specific pathogenic CD4+ T cells using an epitope-MHC Class II tetramer construct to enrich and label the allergen-specific pathogenic CD4+ T cells. In contrast, the present example demonstrates that pathogenic CD4+ T_{H}2 cells only present in allergic individuals (T_{H}2A cells) can be identified through ST2 detection rather than requiring detection of a combination of cell surface biomarkers. T_{H}2A cells selectively express the IL-33 receptor, ST2, upon allergen exposure. This expression requires T cell receptor (TCR) triggering, which occurs via MHC class II presentation of allergen peptides by antigen presenting cells (APCs) to T cells. ST2 expression does not occur in non-atopic individuals, nor does it occur when T cells from atopic individuals are cultured in the presence of an allergen for which the individual has no allergy. These results not only demonstrate an improved method for identifying and assessing allergen-reactive T_{H}2A cells, but also indicate the ST2 biomarker can be used clinically to differentiate allergic individuals from non-allergic individuals.

Because an increase in CD27+ T cells and a decrease in T_{H}2A cells is typically observed during allergen-specific immunotherapy (ASIT) in individuals who respond to the therapy, ST2 expression status can be used as a biomarker indicating response to ASIT.

### Results

The ex vivo pMHCII tetramer approach was used to characterize allergen-specific pathogenic CD4+ T cells expressing ST2 in patients with food allergies (e.g., peanut, walnut), perennial allergies (e.g., house dust mite), seasonal pollen allergies (e.g., Alder tree, grass), or chemical allergies (e.g., pool inhalant). Non-allergic individuals were used as a control group. Regardless of the allergen tested in this study, IgE-mediated allergic diseases were characterized by high frequencies of allergen-specific CRT_{H}2+ T cells, which were strictly absent in non-allergic subjects, suggesting that presence of these CD4+ effector T cells is necessary for allergy pathogenesis. ST2, the IL-33 receptor, was up-regulated within the T_{H}2A cell subset during a patient's exposure to an allergen to which they were allergic. Collectively, the results demonstrate that the T_{H}2A cell subset represents a phenotypically distinct T_{H}2 subpopulation, which can encompass the vast majority of pathogenic T_{H}2 cells involved in type I allergic diseases.

### Example 2

### Abstract

This example describes methods of detecting, separating, and/or monitoring the presence of pro-allergic T cells to a particular allergen of interest in a subject. The exemplary method does not rely on epitope-MHC tetramers to identify the state of T cell activation in an allergen specific manner. In the exemplary method, allergen-specificity is determined by selective expression of the ST2 molecule (IL-33 receptor) following a stimulation with an allergen of interest. As described below, this single screen for activation markers, independent of epitope binding (e.g., not using a peptide epitope/MHCII tetramer), successfully identifies activated allergen-specific T_{H}2 subpopulations that are pathogenic and indicative of allergen sensitivity in the subject. Accordingly, the disclosed screen is useful for methods including:
detecting and quantifying pro-allergic (i.e., pathogenic) allergen-specific T cells;
isolating and purifying live pro-allergic allergen-specific T cells;
assessing the clinical status (e.g., “Allergic” vs. “Non-allergic”) of the patient;
determining achievement of clinical benefit in allergic patient receiving immunotherapy (e.g., monitoring immunotherapy); and
testing effect of drug compound on live pro-allergic allergen-specific T cells.

### Background

Currently, allergen-specific immunotherapy (ASIT) is the only known disease-modifying treatment of allergic diseases. The principle of ASIT is to administer gradually increasing doses of allergen, either as an allergen extract or as recombinant allergen. While evidence to date has revealed that ASIT can be clinically efficacious, a long period of time may elapse before achievement of clinical benefit and a subset of patients appears to be clinically unresponsive to ASIT. In the absence of information about primary clinical endpoints, biomarkers can provide critical insights that allow investigators to guide the clinical development of the candidate vaccine. However, assumptions about a correlation between immunological end-points and clinical outcomes of allergy vaccine are not supported by current monitoring strategies. Given their pivotal role in both the induction and control of allergic inflammation, quantifiable changes at the level of CD4+ T cells could represent a clinically meaningful signature that will reflect and quantify an underlying allergic disease process. This, in turn, would facilitate the monitoring of clinical outcomes, facilitate design and evaluation of allergy vaccines, and enable our understanding of mechanisms of action associated with successful immunotherapy.

The recent use of antigen-specific T cell assay in the allergy context demonstrated that allergen-specific CD4+ memory T_{H}2 cells are correlated with their functional activities and sensitivity to ASIT. These data have several important implications for understanding the basic immunologic mechanisms involved in the amelioration of allergic symptoms during allergen-SIT. First, it demonstrates that allergic and non-allergic individuals have functionally and phenotypically distinct circulating subsets of allergen-specific CD4+ T cells, which can be clearly differentiated based on their ST2 expression. Finally, successful ASIT leads to selective elimination of allergen-specific T_{H}2 cells in the peripheral blood. These data suggest a novel mechanism in which the depletion of allergen-specific T_{H}2 cells might be a prerequisite for the induction of specific tolerance. Importantly, this could lead to the development of predictive markers for the clinical success of ASIT but also to new vaccine strategies to enhance the power of allergen-specific immunotherapy. In this context, monitoring ST2 expression on antigen-specific T cells provide a valuable tool for assessing the clinical status (Allergic vs. Non-allergic) of the patient to a particular allergen and for determining achievement of clinical benefit in patient receiving allergen immunotherapy.

### Study Overview

To establish a reliable method to ascertain the presence of pathogenic T cells specific for an allergen, a protocol was developed that incorporated a screen for an activation marker (ST2), which is independent of the functional immune receptors that bind to the allergen itself. The protocol was applied to characterize the subpopulations of allergen-specific T_{H}2 cells from individuals that were allergic or not allergic to an allergen, as well as monitor the efficacy of immunotherapy in subjects that were allergic to an allergen.

### Screen Protocol

1. PBMCs were isolated from whole blood using density gradient centrifugation.

2. PBMCs (0.5-10×10⁷ cells) were then stimulated for 1 to 48 hrs in RPMI 1640 supplemented with 5% AB serum, with the antigen (e.g., allergen-derived peptides, protein, or allergen crude extract) at 37° C. and 5% CO₂.

3. After stimulation, cells were centrifuged at 1200 RPM for 5 minutes.

4. Enriching antibodies (α-ST2) were added to cells at a volume of 10 μl per million cells.

5. Cells were vortexed and resuspended and allowed to incubate for 20 minutes at 37° C. in the dark.

6. Cells were washed with 4 ml of PBS and centrifuged at 1200 RPM for 5 minutes and decanted.

7. 35 μl of Miltenyi magnetic microbeads were added to each tube per million cells. Cells were then resuspended and vortexed, and allowed to incubate for 10-15 minutes at RT in the dark.

8. Cells were washed with 4 ml of PBS and centrifuged at 1200 RPM for 5 minutes and decanted.

9. During step 6, 1 ml of PBS was added to the appropriate number of Miltenyi MS columns loaded onto a magnet. Flow through was collected in a new falcon tube.

10. Cells were resuspended in 1 ml of PBS per 50 million cells. Resuspended cells were added to a Miltenyi MS column and allow to completely pass through column while the flow through was collected in the same falcon tube as step 7.

11. Another 1 ml of PBS was added to the MS column and three drops were allowed to fall into the flow through tube while still attached to the magnet. After three drops, the column was transferred to a new falcon tube and the remaining volume of PBS was allowed to flow through the column.

12. After column the column was eluted, 1 ml of PBS was added to the liberated column and plunged at a rate of 2 drops/second into the elution falcon tube.

13. Elution column was centrifuged at 1200 RPM for 5 minutes and decanted.

14. Surface staining antibody cocktail (including at minimum CD4) was then added to remaining volume of cells and allowed to incubate for 15-20 minutes at RT in the dark.

15. Washed cells were resuspended in 200 μl of PBS and analyzed via flow cytometry.

The described protocol can be applied to cells in a variety of sample formats, including running the protocol directly on whole blood instead of on isolated PBMCs. Furthermore, samples can be frozen prior to the protocol.

Allergen challenge is not limited to any particular allergen or format. The allergen can be presented in purified form, in crude extract, as whole allergen, as allergen fragment, or as epitope in MHC.

Enrichment or isolation of the activated allergen-specific T cells from the PBMCs, e.g., starting at step 4, above, is optional and not required for the ultimate detection and quantification of activated allergen-specific T_{H}2 cells.

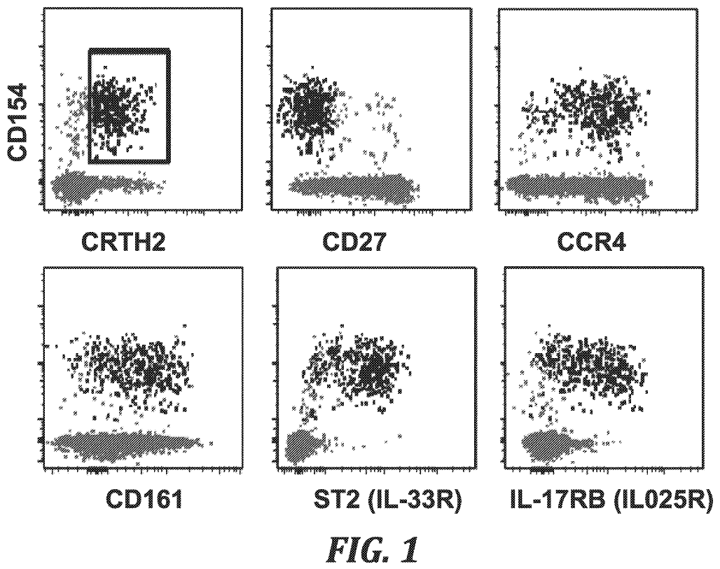

FIG. 1 shows the expression of the markers CRT_{H}2, CD27, CCR4, CD161, ST2, and IL-17RB. The data show that the pathogenic T_{H}2 cells are CRT_{H}2+, CD27−, CCR4+, CD161+, ST2+ and IL-17RB+. The data also show that pathogenic T_{H}2 cells can be selected based on the presence of ST2.

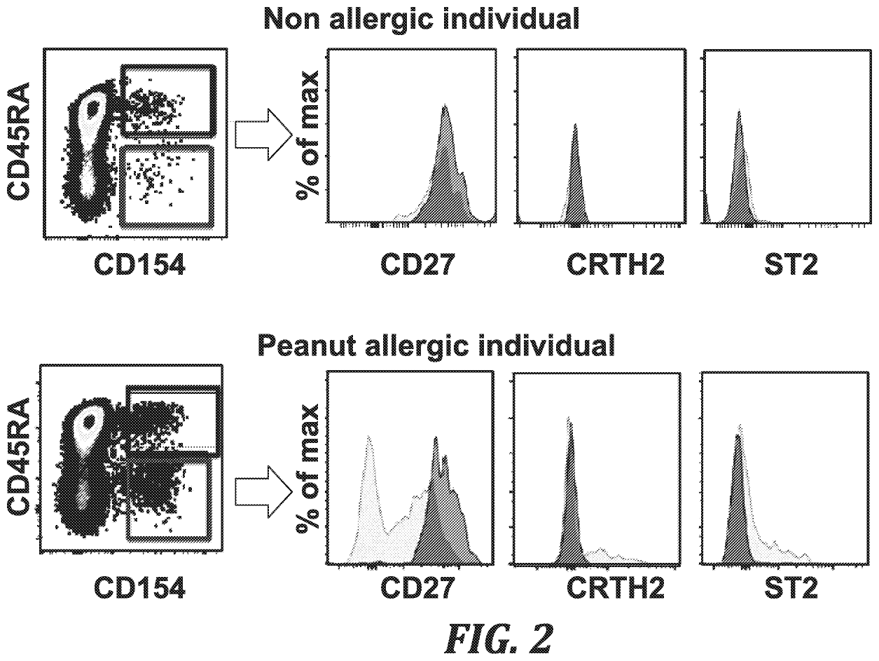

FIG. 2 demonstrates the presence of CD27− and CRT_{H}2+ T cells in allergic individuals, and the absence of CD27− and CRT_{H}2+ T cells in non-allergic individuals. FIG. 2 also illustrates ST2 expressing allergen-specific pathogenic CD4+ T cells are confined to atopic individuals.

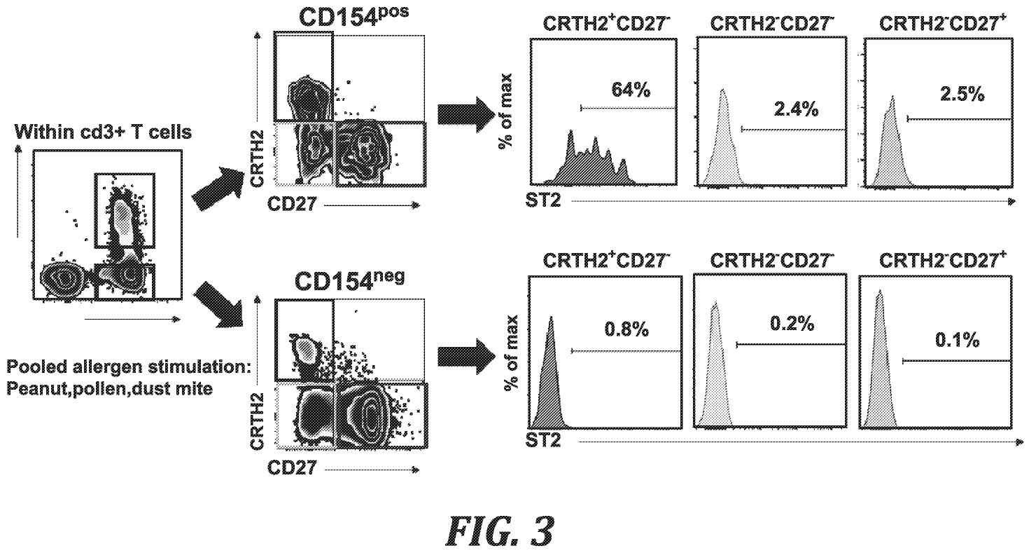

FIG. 3 illustrates that ST2 expression on T cells is confined to the allergen-reactive CD4+ subset. The data also show that ST2 expression on human T_{H}2A cells requires prior T cell receptor triggering.

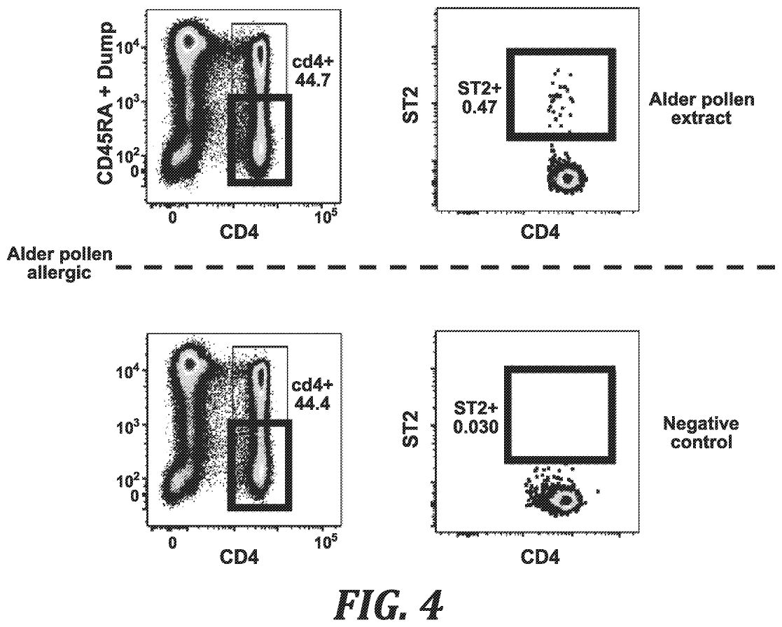

FIG. 4 demonstrates that ST2-expressing T cells are only present when PBMCs from an allergic individual are contacted with the allergen to which the individual is allergic.

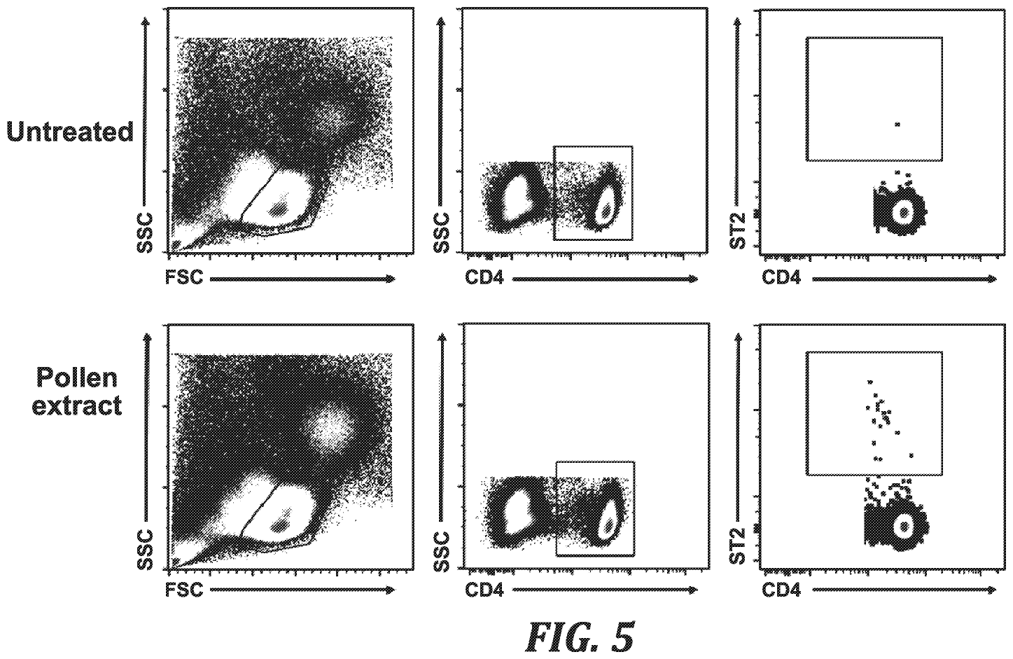

FIG. 5 demonstrates that ST2-expressing T cells are only present when PBMCs from an allergic individual are contacted with the allergens (pollen) to which the individual is allergic.

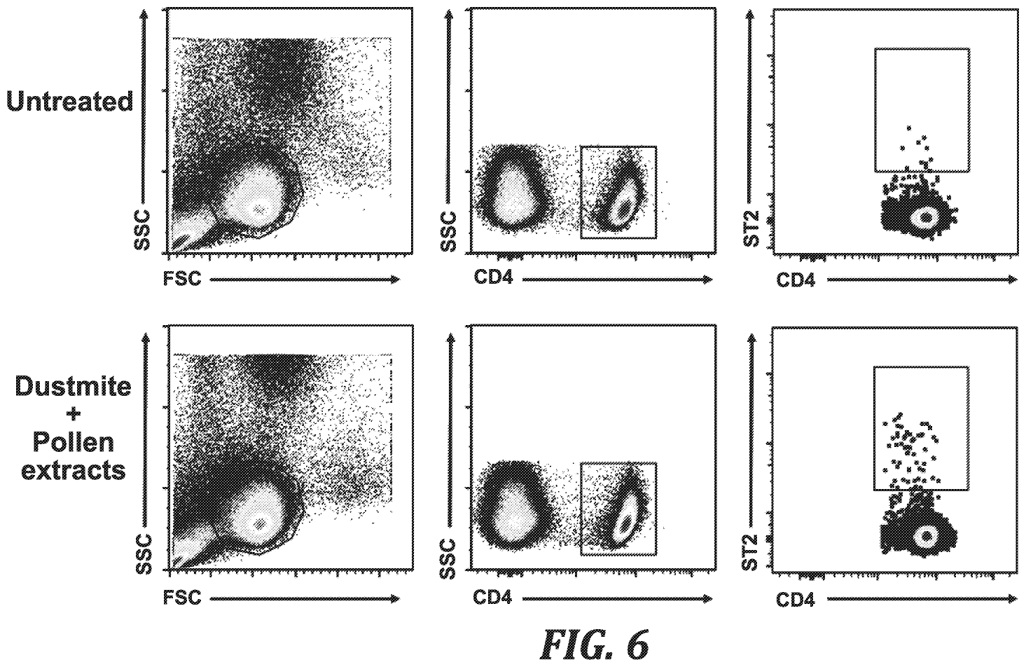

FIG. 6 demonstrates that ST2-expressing T cells are only present when PBMCs from an allergic individual are contacted with the allergens (pollen and dust mite) to which the individual is allergic.

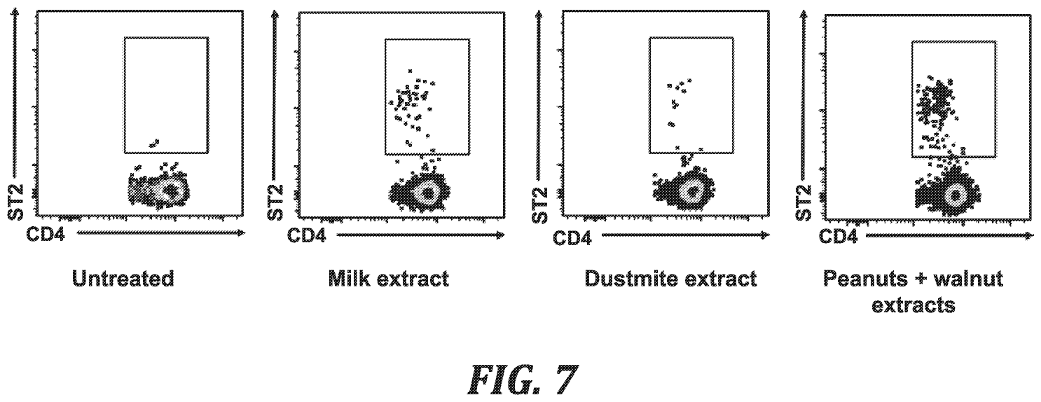

FIG. 7 demonstrates that ST2-expressing T cells are only present when PBMCs from an allergic individual are contacted with the allergens to which the individual is allergic.

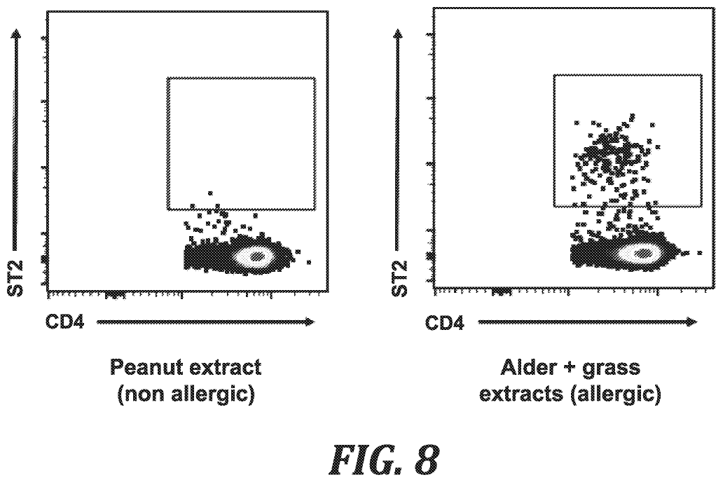

FIG. 8 demonstrates that ST2-expressing T cells are only present when PBMCs from an allergic individual are contacted with the allergen to which the individual is allergic. FIG. 8 also demonstrates that ST2-expressing T cells are not present when PBMCs from an allergic individual are contacted with allergens the individual is not allergic to, nor are they present when T cells undergo a polyclonal, nonspecific activation via the CD3/CD28 pathway.

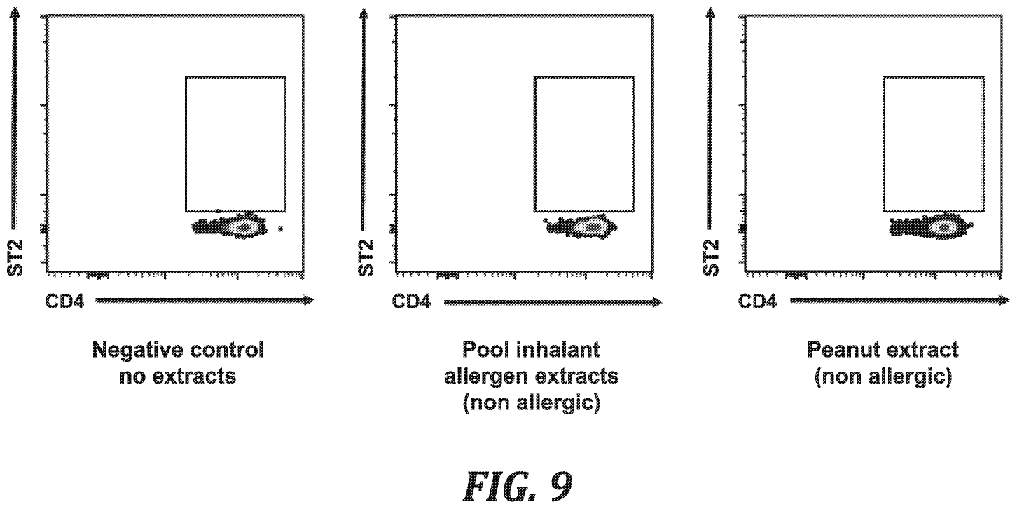

FIG. 9 demonstrates that ST2-expressing T cells are not present when PBMCs from a non-allergic individual are contacted with known allergens of differing types (inhalant vs food) or differing preparations (crude extract vs allergen peptides).

### Example 3

### Exemplary Methods and Materials

T_{H} Cell Stimulation and Isolation

Isolated PBMCs, or previously frozen and thawed PBMCs, were isolated from whole blood (5-10mL) or tissue, using density gradient centrifugation. PBMCs were then stimulated with crude allergen extract or peptides (18hr in RPMI 1640 supplemented with 5% AB serum, at 37° C. and 5% CO₂). After stimulation, cells were harvested by centrifugation (400 rcf for 5 minutes), washing (1× PBS), centrifugation, and decanting PBS supernatant. Cells were stained with PE-anti ST2 antibody (20μl diluted to 100 μl with 1× PBS for between 10⁶ and 10⁷ cells, or 10 μl antibody diluted to 100 μl 1×PBS per 1 million cells). The mixture was vortexed gently, incubated (30 minutes at 37° C.), washed with PBS, and centrifuged (400 rcf for 5 minutes), decanting as much PBS as possible afterward. To the mixture, PBS (65μL) and Miltenyi anti-PE microbeads (35 μL) were added to each tube to enrich the sample. This enrichment step is optional. The mixture was gently vortexed and incubated in the dark (10 minutes at RT). The sample was washed with PBS, centrifuged (400 rcf for 5 minutes), and the PBS decanted. During this step, PBS (1 mL) was added to the Miltenyi MS columns loaded onto a magnet with flow-through collected in a new Falcon tube. After centrifugation, the cells were resuspended (1 mL of PBS) and added to a Miltenyi MS column. The sample was allowed to completely pass through the column while the flow-through was collected in the previous flow-through Falcon tube. Additional PBS (1 mL) was added to the MS column and two drops were allowed to fall into the flow through tube while the sample was still attached to the magnet. After elution of the second drop, the column was rapidly transferred to a new Falcon tube and the remaining PBS volume allowed to flow through the column. Additional PBS (1 mL) was added to the liberated column and plunged into the elution Falcon tube constituting of ST2+ enriched cells. The ST2+enriched cell eluent was centrifuged (400 rcf for 5 minutes), and the supernatant decanted.

Surface Stain Enriched Cells for CD4

Each tube from the T_{H} cell isolation procedure received anti-CD4 (2 μL) diluted to 100 μL with PBS. Additional surface biomarkers can be added as desired, maintaining the 100 μL total volume. After extracellular staining, intracellular staining can commence. For desired intracellular staining, Foxp3 transcription factor buffer set was used according to kit directions. The sample was incubated (25 minutes at RT in the dark), washed (PBS), centrifuged (400 rcf for 5 minutes), and the PBS supernatant decanted.

Determining Allergic Status

The main objective of this study was to determine whether ST2 expression on T_{H}2 cells was associated with global atopic inflammatory disorders. To investigate allergy-related differences in peripheral T cells from allergic individuals, the profile of allergen-specific T_{H}2 cells was determined ex vivo using direct pMHCII tetramer staining, and compared with the profile of a ST2-expressed T_{H}2 cell subset. Candidate signature ST2 biomarkers were then tested in allergic patients and in non-atopic individuals. The presence of a ST2-expressed CD4+ T cell indicates the subject is allergic to the allergen, and the absence of a ST2-expressed CD4+ T cell indicates the subject is either not allergic or has a degree of sensitization to the allergen.

Monitoring Immunotherapy Efficacy

Allergen-specific T_{H}2 cells can be measured in a biological sample obtained from a patient during or following a maintenance phase of an immunotherapy to monitor treatment. A patient can be treated for an allergy by administering to the patient an effective amount of an allergy immunotherapy composition, and monitoring the treatment of the patient by measuring the allergen-specific T_{H}2 cells in a biological sample obtained from the patient during the treatment.

### Conclusion

These results described herein demonstrate that the single screen for an activation marker (e.g., ST2) can accurately detect activated allergen-specific T_{H}2 cells that are pathogenic and cause sensitivity of the subject to the allergen in question. Specifically, the cells expressing ST2 are shown to be pathogenic activated allergen-specific T_{H}2 cells and, thus, their presence indicates sensitivity of the subject to the allergen. Subjects receiving immunotherapy for the allergen demonstrate a marked reduction in ST2+ T cells which is associated with a simultaneous reduction in sensitivity. The presence of ST2+ T cells can be used as a target to detect sensitivity to allergens and monitor therapies thereto. As demonstrated here, the disclosed screen can monitor the presence of such pathogenic T cells and can facilitate monitoring of their reduced presence during treatment as an indicator of the efficacy of the treatment.

### Exemplary embodiments

A1. A method of specifically labeling an allergen-specific pathogenic CD4+ T cell, comprising:

contacting a cell population comprising CD4+ T cells with a suspected allergen to provide a challenged cell population,

contacting the challenged cell population, or a subpopulation thereof, with a first molecule that specifically binds to a biomarker for an allergen-specific pathogenic T cell, and

detecting binding of the first molecule to a CD4+ cell.

A2. The method of embodiment A1, wherein binding to the cell indicates the cell is an allergen-specific pathogenic CD4+ T cell.

A3. The method of embodiment A1 or A2, wherein the cell population comprises cells derived from a tissue sample obtained from a subject.

A4. The method of embodiment A1 or A2, wherein the cell population comprises whole blood or peripheral blood mononuclear cells (PBMCs) obtained from a subject.

A5. The method of embodiments A1-A4, wherein the suspected allergen comprises a crude allergen extract, a peptide, an allergen protein, or any combination thereof, or is part of a food-derived mixture.

A6. The method of embodiments A1-A5, wherein the biomarker is ST2 and detection of binding of the first molecule to ST2 is indicative of an allergen-specific pathogenic CD4+ T cell.

A7. The method of embodiments A1-A5, wherein the biomarker is IL-17RB and detection of binding of the first molecule to IL-17RB is indicative of an allergen-specific pathogenic CD4+ T cell.

A8. The method of embodiments A1-A7, wherein the first molecule is selected from an antibody, antibody-like molecule, receptor, aptamer, or a functional antigen-binding fragment or derivative thereof

A9. The method of embodiment A8, wherein the antibody-like molecule is a single-chain antibody, a bispecific antibody, a Fab fragment, or a F(ab)₂ fragment.

A10. The method of embodiment A9, wherein the single-chain antibody is a single chain variable fragment (scFv), single-chain Fab fragment (scFab), V_{H}H fragment, V_{NAR}, or nanobody.

A11. The method of embodiments A1-A10, wherein the first molecule comprises a detectable label.

A12. The method of embodiment A11, wherein the detectable label is fluorescent, a heavy metal moiety, or an oligonucleotide conjugate.

A13. The method of embodiments A1-A12, further comprising enriching for the allergen-specific pathogenic CD4+ T cells.

A14. The method of embodiments A1-A13, further comprising isolating a subpopulation of allergen-specific pathogenic CD4+ T cells.

A15. The method of embodiment A14, wherein enriching or isolating comprises the use of magnetic beads or flow cytometry.

A16. The method of embodiments A1-A15, wherein detection of binding of the molecule to the biomarker comprises use of Fluorescence-activated cell sorting (FACS), mass cytometry (CyTOF), qPCR, mass spectrometry, or microscopy.

A17. The method of embodiment A16, wherein the cells are analyzed by flow cytometry, confocal microscopy, qPCR, or mass spectrometry.

A18. The method of embodiments A3-A4, further comprising obtaining the whole blood, PBMCs, or tissue from the subject.

A19. The method of embodiment A2, wherein a determined presence of at least one allergen-specific pathogenic CD4+ T cell biomarker is indicative that the subject is allergic with respect to the suspected allergen.

B1. A method of determining whether a subject is allergic to a suspected allergen, comprising:

contacting tissue, whole blood, or peripheral blood mononuclear cells (PBMCs) obtained from a subject with a suspected allergen to provide challenged PBMCs,

contacting the challenged PBMCs, or a subpopulation thereof, with a first molecule that specifically binds to a biomarker for an allergen-specific pathogenic CD4+ T cell,

detecting binding of the first molecule to a CD4+ cell, wherein binding to the cell indicates the cell is an allergen-specific pathogenic CD4+ T cell, and

determining the presence of an allergen-specific pathogenic CD4+ T cell, wherein the presence of an allergen-specific pathogenic CD4+ T cell indicates the subject is allergic to the allergen, and the absence of an allergen-specific pathogenic CD4+ T cell indicates the subject is either not allergic or has a degree of sensitization to the suspected allergen.

B2. The method of embodiment B1, further comprising treating the subject's allergic condition.

B3. The method of embodiment B2, wherein treating the subject comprises administering immunotherapy.

C1. A method of monitoring the presence of allergen-specific pathogenic CD4+ T cells in a subject allergic to an allergen, comprising:

performing the following steps at two or more time points:
contacting tissue, whole blood, or peripheral blood mononuclear cells (PBMCs) obtained from the subject with the suspected allergen to provide challenged PBMCs,
contacting the challenged PBMCs, or a subpopulation thereof, with a first molecule that specifically binds to a biomarker for an allergen-specific pathogenic CD4+ T cell,
detecting binding of the first molecule to a CD4+ cell, wherein binding to the cell indicates the cell is an allergen-specific pathogenic CD4+ T cell, and

determining the relative abundance over time of an allergen-specific pathogenic CD4+ T cell, wherein a decreased abundance of the allergen-specific pathogenic CD4+ T cell indicates the subject has become less allergic to the allergen.

C2. The method of embodiment C1, wherein at least one of the two or more time points is during or after treatment of the subject's allergic condition.

C3. The method of embodiments C1-C2, wherein an absence of allergen-specific pathogenic CD4+ T cells indicates the subject is either no longer allergic or has a degree of desensitization to the allergen.

D1. A method of monitoring the efficacy of the immunotherapy of a subject allergic to an allergen, comprising the following at one or more time points during the subject's immunotherapy:

contacting tissue, whole blood, or peripheral blood mononuclear cells (PBMCs) obtained from a subject with a suspected allergen to provide challenged PBMCs,

contacting the challenged PBMCs, or a subpopulation thereof, with a first molecule that specifically binds to a biomarker for an allergen-specific pathogenic CD4+ T cell,

detecting binding of the first molecule to a CD4+ cell, wherein binding to the cell indicates the cell is an allergen-specific pathogenic CD4+ T cell, and

determining the relative abundance over time of an allergen-specific pathogenic CD4+ T cell, wherein a decreased abundance of allergen-specific pathogenic CD4+ T cells indicates efficacy of the immunotherapy.

E1. The method of embodiments B1-B3, C1-C3, and D1, wherein the method comprises obtaining PBMCs from the subject.

E2. The method of embodiments B1-B3, C1-C3, and D1, wherein the method comprises enriching for, or isolating, the allergen-specific pathogenic CD4+ T cells.

E3. The method of embodiment E2, wherein enriching or isolating comprises the use of flow cytometry or magnetic beads.

E4. The method of embodiments B1-B3, C1-C3, D1, and E1-E3, wherein the biomarker is ST2 and detection of binding of the first molecule to ST2 is indicative of an allergen-specific pathogenic CD4+ T cell.

E5. The method of embodiments B1-B3, C1-C3, D1, and E1-E3, wherein the biomarker is IL-17RB and detection of binding of the first molecule to IL-17RB is indicative of an allergen-specific pathogenic CD4+ T cell.

E6. The method of embodiments B1-B3, C1-C3, D1, and E1-E5, wherein the first molecule is selected from an antibody, antibody-like molecule, receptor, aptamer, or a functional antigen-binding fragment and domain thereof.

E7. The method of embodiment E6, wherein the antibody-like molecule is a single-chain antibody, a bispecific antibody, a Fab fragment, or a F(ab)₂ fragment.

E8. The method of embodiment E7, wherein the single-chain antibody is a single chain variable fragment (scFv), single-chain Fab fragment (scFab), V_{H}H fragment, V_{NAR}, or nanobody.

E9. The method of embodiments B1-B3, C1-C3, D1, and E1-E8, wherein the first molecule comprises a detectable label.

E10. The method of embodiment E9, wherein the detectable label is fluorescent, a heavy metal moiety, or an oligonucleotide conjugate.

F1. A kit, comprising a molecule that specifically binds to a biomarker for an allergen-specific pathogenic CD4+ T cell.

F2. The kit of embodiment F1, wherein the molecule is an antibody, antibody-like molecule, ligand, aptamer, or a functional antigen-binding fragment or derivative thereof.

F3. The kit of embodiment F2, wherein the antibody-like molecule is a single-chain antibody, a bispecific antibody, a Fab fragment, or a F(ab)₂ fragment.

F4. The kit of embodiment F3, wherein the single-chain antibody is a single chain variable fragment (scFv), single-chain Fab fragment (scFab), V_{H}H fragment, V_{NAR}, or nanobody.

F5. The kit of embodiments F1-F4, wherein the molecule specifically binds to ST-2 (IL-33R).

F6. The kit of embodiments F1-F4, wherein the molecule specifically binds to IL-17RB (IL-25R).

While illustrative embodiments have been illustrated and described, it will be appreciated that various changes can be made therein without departing from the spirit and scope of the invention.

1. A method of specifically labeling an allergen-specific pathogenic CD4+ T cell, comprising:
contacting a cell population comprising a CD4+ T cell with a suspected allergen to provide a challenged cell population,
contacting the challenged cell population, or a subpopulation thereof, with a first molecule that specifically binds to a biomarker for an allergen-specific pathogenic T cell, and
detecting binding of the first molecule to the CD4+ cell.

2. The method of claim 1, wherein binding to the CD4+ cell indicates the cell is an the allergen-specific pathogenic CD4+ T cell.

3. The method of claim 1, wherein the cell population comprises cells derived from a tissue sample obtained from a subject.

4. The method of claim 1, wherein the cell population comprises whole blood or peripheral blood mononuclear cells (PBMCs) obtained from a subject.

5. The method of claim 1, wherein the suspected allergen comprises a crude allergen extract, a peptide, an allergen protein, or any combination thereof, or is part of a food-derived mixture.

6. The method of claim 1, wherein the biomarker is ST2 and detection of binding of the first molecule to ST2 is indicative of the allergen-specific pathogenic CD4+ T cell.

7. The method of claim 1, wherein the biomarker is IL-17RB and detection of binding of the first molecule to IL-17RB is indicative of the allergen-specific pathogenic CD4+ T cell.

8. The method claim 1, wherein the first molecule is selected from an antibody, antibody-like molecule, receptor, aptamer, or a functional antigen-binding fragment or derivative thereof

9. The method of claim 8, wherein the antibody-like molecule is a single-chain antibody, a bispecific antibody, a Fab fragment, or a F(ab)₂ fragment.

10. The method of claim 9, wherein the single-chain antibody is a single chain variable fragment (scFv), single-chain Fab fragment (scFab), V_{H}H fragment, V_{NAR}, or nanobody.

11. The method of claim 1, wherein the first molecule comprises a detectable label.

12. (canceled)

13. The method of claim 1, further comprising enriching for the allergen-specific pathogenic CD4+ T cells.

14. (canceled)

15. The method of claim 13, wherein enriching comprises the use of magnetic beads or flow cytometry.

16. The method of claim 1, wherein detection of binding of the molecule to the biomarker comprises use of Fluorescence-activated cell sorting (FACS), mass cytometry (CyTOF), qPCR, mass spectrometry, or microscopy.

17-18. (canceled)

19. The method of claim 2, wherein a determined presence of at least one allergen-specific pathogenic CD4+ T cell biomarker is indicative that the subject is allergic with respect to the suspected allergen.

20. A method of determining whether a subject is allergic to a suspected allergen, comprising:
contacting tissue, whole blood, or peripheral blood mononuclear cells (PBMCs) obtained from a subject with a suspected allergen to provide challenged PBMCs,
contacting the challenged PBMCs, or a subpopulation thereof, with a first molecule that specifically binds to a biomarker for an allergen-specific pathogenic CD4+ T cell,
detecting binding of the first molecule to a CD4+ cell, wherein binding to the cell indicates the cell is the allergen-specific pathogenic CD4+ T cell, and
determining the presence of the allergen-specific pathogenic CD4+ T cell, wherein the presence of the allergen-specific pathogenic CD4+ T cell indicates the subject is allergic to the allergen, and the absence of the allergen-specific pathogenic CD4+ T cell indicates the subject is either not allergic or has a degree of sensitization to the suspected allergen.

21. The method of claim 20, further comprising treating the subject's allergic condition.

22. The method of claim 21, wherein treating the subject comprises administering immunotherapy.

23. A method of monitoring the presence of allergen-specific pathogenic CD4+ T cells in a subject allergic to an allergen, comprising:
performing the following steps at two or more time points:
contacting tissue, whole blood, or peripheral blood mononuclear cells (PBMCs) obtained from the subject with a suspected allergen to provide challenged PBMCs,
contacting the challenged PBMCs, or a subpopulation thereof, with a first molecule that specifically binds to a biomarker for the allergen-specific pathogenic CD4+ T cell,
detecting binding of the first molecule to a CD4+ cell, wherein binding to the cell indicates the cell is the allergen-specific pathogenic CD4+ T cell, and
determining the relative abundance over time of the allergen-specific pathogenic CD4+ T cell, wherein a decreased abundance of the allergen-specific pathogenic CD4+ T cell indicates the subject has become less allergic to the allergen.

24. The method of claim 23, wherein at least one of the two or more time points is during or after treatment of the subject's allergic condition.

25-39. (canceled)
